(19) United States
(12) Patent Application Publication
(10) Pub. No.: US 2022/0370390 A1
(43) Pub. Date: Nov. 24, 2022

(54) LIQUID ORAL FORMULATION OF BUMETANIDE

(71) Applicant: NEUROCHLORE, Marseille Cedex 09 (FR)

(72) Inventors: Yehezkel BEN-ARI, La Ciotat (FR); Denis RAVEL, Paris (FR); Gérard DAMIEN, Meung sur loire (FR)

(73) Assignee: NEUROCHLORE, Marseille Cedex 09 (FR)

(21) Appl. No.: 17/773,201

(22) PCT Filed: Oct. 31, 2020
(86) PCT No.: PCT/EP2020/080612

§ 371 Date: Apr. 29, 2022

Related U.S. Application Data
(60) Provisional application No. 62/928,665, filed on Oct. 31, 2019.

(51) Int. Cl. A61K 31/196; A61K 47/14; A61K 47/54

(52) U.S. Cl. CPC A61K 31/196; A61K 47/14; A61K 47/548; A61K 47/549

(57) ABSTRACT

The invention relates to a liquid oral formulation of bumetanide for the treatment of autism, more particularly for the improvement of the Autism Spectrum Disorder (ASD) core symptoms, tuberous sclerosis complex, fragile X syndrome, Rett syndrome, Down syndrome, cancer and particularly gliomas, spinal cord lesions, chronic pain, brain trauma, cerebrovascular infarcts, various types of epilepsies, and also acute lung injury such as pneumonias or Severe Acute Respiratory Disease due to Coronavirus-2 (SARS-CoV-2). Said formulation is especially designed for paediatric populations. The invention relates also to a posology for the administration of said liquid oral formulation.

The invention relates to a liquid oral formulation of bumetanide for the treatment of autism, more particularly for the improvement of the Autism Spectrum Disorder (ASD) core symptoms; said formulation is especially designed for paediatric populations. The invention relates also to a posology for the administration of said liquid oral formulation.

Bumetanide or 3-(Butylamino)-4-phenoxy-5-sulfamoylbenzoic acid (IUPAC name) is known as a loop diuretic with good absorption and pharmacodynamic properties (Ramsay et al., Br. J. Clin. Pharmacol. 5(3), 243-247 (1978)). It acts as an inhibitor of the Na—K—Cl cotransporter 1 (NKCC1) and the kidney-specific NKCC2 (Horster et al., Am. J. Physiol. Renal Physiol. 279(6), 982-996 (2000); Gillen et al., J. Biol. Chem. 271(27), 16237-16244 (1996)) and also inhibits several isoforms of carbonic anhydrases (Temperini et al., Bioorg. Med. Chem. Lett. 18(8), 2567-2573 (2008)). Bumetanide is a widely used, highly potent loop diuretic that inhibits Na+ and Cl— reabsorption by the thick ascending limb of the loop of

Henle by blocking the Na+-K+-Cl— cotransporter isoform 2 (NKCC2) located in the apical membrane of these epithelial cells, (Haas and Forbush, The Na—K—Cl cotransporters. J Bioenerg Biomembr. 1998; 30:161-172). This results in excretion of sodium, chloride, and water and, hence, diuresis. In the brain, bumetanide inhibits the Na+-K+-Cl— cotransporter isoform 1 (NKCC1). This results in inhibiting chloride uptake in Gabaergic neurones thus, decreasing the excitatory effect of GABA in neonates and several pathologic condition in children and adults (Lemonnier et al. Translational Psychiatry (2017) 7).

Bumetanide is already used for the treatment of oedema associated with congestive heart failure, hepatic and renal disease including the nephrotic syndrome. For said treatments, the approved formulations are tablets of 0.5 mg, 1.0 mg, 2.0 mg or 5.0 mg and liquid for injection at 0.25 mg/ml and 0.5 mg/ml. An oral solution of 0.2 mg/ml of bumetanide was also developed and marketed for use when diuretic therapy is required in the treatment of oedema; said solution is not recommended for children. The posology of this oral solution is 1 mg as a single dose given morning or early evening. The excipients in said oral solution are standard excipients for masking the taste and the colour of bumetanide and for stabilizing the drug in order to be stored during the treatment.

The patent EP3004353 provides that bumetanide can be used in the treatment of neurodegenerative diseases with Parkinsonian syndromes, in combination or not with other active agent(s) for treating Parkinsonian syndromes and/or side effects of said active agent(s). The posology consists in a dose from about 0.01 mg to about 500 mg per day, preferably from about 0.1 mg to about 5 mg per day. The route of administration is injection, preferably systemically, or oral administration via a solid form, a liquid form or a gel.

Several studies show an interesting effect of bumetanide in the treatment of cancer. The bumetanide was demonstrated to have an effect on gliomas that are the most prevalent type of intracranial tumors. We can cite as example the use of bumetanide as an inhibitor of NKCC1 (NA+K+Cl— cotransporter isoform1) in the treatment of glioblastoma cancer cells

(Habela et al., J Neurophysiol. 2009 Feb;101(2):750-7—Algharabli et al., Cell Physiol Biochem . 2012; 30(1): 33-48.), in the glioma apoptosis and astrogliosis reduction (Luo et al., Mol Cancer Ther May 11, 2020; DOI: 10.1158/1535-7163). Importantly bumetanide attenuates the severity of glioblastoma and the expression of the NKCC1 cotransporter that bumetanide blocks is also directly correlated with the severity of glioblastoma grade and is abolished or strongly reduced in grafts of human glioblsatoma in NKCC1 Kos (Garzon-Muvdi et al., PLoS Biol. 2012 May; 10(5): e1001320). The bumetanide has also a significant effect in a colon cancer model (Malamas et al. Pharm Res. 2015 Sep.; 32(9):3029-3043) and on hepatocellular carcinomas (Feng et al., Oncotarget. 2016 Aug. 16;7(33):53571-53582).

Several studies show a significant effect of bumetanide in the treatment of acute lung injury by suppressing macrophage activation (Chin-Mao Hung et al., Biochemical Pharmacology 156 (2018) 60-67) and also on the effects on the pulmonary system during acute inflammation such as bacterial pneumonia (Nguyen et al., J. Exp. Med. 2007 Jun 11;204(6):1383-93). During the treatment with bumetanide an increase in the alveolar fluid clearance is observed; it is known that alveolar fluid clearance driven by active epithelial Na⁺ and secondary Cl^{—} absorption counteracts edema formation in the intact lung and that impairment of alveolar fluid clearance promotes cardiogenic lung edema (Solymosi et al., PNAS 2013 Jun. 18;110(25):E2308-16) and also that the impaired fluid clearance correlates strongly with increased mortality of patients with acute respiratory distress syndrome (Ware et al., Am J Respir Crit Care Med. 2001 May;163(6):1376-83). In this study the bumetanide administered intrapulmonary reduces the severity of the inflammation-related induced acute lung injury since it reduces cell volume and inhibits the functions of alveolar macrophages. Consequently it seems that other pathologies such as potentially lethal infections caused by the novel Severe Acute Respiratory Disease Coronavirus-2 (SARS-CoV-2) could also be treated with bumetanide (Brennecke et al., Amer. Jour. Of the Med. Sciences Volume 360, Issue 3, Sep. 2020, Pages 216-221) since its effect on cell volume and alveolar macrophages could attenuate the effects and/or symptoms of the disease for a patient in need thereof

The patent EP2523661 shows that bumetanide can also be used in the treatment of autism and particularly in the improvement of autism spectrum disorder (ASD) core symptoms. Infantile Autistic Syndrome Disorders (ASD) include a wide range of abnormalities including a genuine incapacity to organise affective relations, behavioural anomalies in reciprocal social interactions, verbal and non-verbal communication, limited interest in the surrounding environment associated with stereotyped movements and repetitive plays (Kanner, 1943; Levy and Hyman, 1993; Levy and Hyman, 2005; Adrien et al., 2001; Blanc et al., 2005; Bourreau et al., 2009). To date ASD can't be cured but may be managed effectively with several types of treatment such as educational and/or behavioural interventions, medication and alternative therapies. Consequently, there is a particular need to develop such medications and also to develop drugs and/or treatments to improve the ASD core symptoms. Recent studies have also shown an effect of bumetanide on neurodevelopmental impairments in patients with tuberous sclerosis complex (Andel et al., Molecular Autism 2020 May 7;11(1):30) that is an autosomal dominant disease that affects multiple organs including the brain and is strongly associated with neurodevelopmental disorders, including autism spectrum disorder symptomatology.

There is also a potential effect of bumetanide on several pathologies and conditions such as spinal cord lesions, chronic pain, brain trauma, cerebrovascular infarcts, Rett and Down syndrome, various types of epilepsies and other genetic and environmental insults (Ben-Ari Y., Trends Neurosci. 2017 Sep.;40(9):536-554). Since bumetanide is a highly specific NKCC1 chloride importer antagonist, bumetanide restores low intracellular Cl⁻ levels and attenuates many disorders in experimental conditions. Various animal models of pathologies have shown an effect of bumetanide on the GABA developmental shift with a maternal administration before birth. In a model of maternal immune activation, it has been shown that bumetanide as an NKCC1 chloride importer antagonist, restores the GABA developmental shift at birth (Fernandez et al., Cereb Cortex. 2019 Aug. 14;29(9):3982-3992). In a model of Rett syndrome it was shown that the GABA developmental shift is abolished at birth and restored by an administration of bumetanide one day before delivery (Lozovaya et al., Sci Rep. 2019 Jun. 25;9(1):9276). And in the valproate and fragile X models of autism, the effect of maternal pretreatment with bumetanide is also demonstrated (Tyzio et al., Science. 2014 Feb. 7;343(6171):675-9). Bumetanide attenuated behavioural alterations with features of autism (vocalisation and interactions with novel rodents) in adult animals (Eftekhari et al., Science 346, 176, 2014). Bumetanide also restored a deficient Gabaergic inhibition in 2 weeks old Fragile X mice model (He Q. et al., Contractor A. J Neurosci. 2014 Jan. 8;34(2):446-50).

In the objective of developing a drug to improve the ASD core symptoms, the inventors based their reflexions on the fact that experimental data suggest that in autism there is a modification in Gaba induced cerebral inhibition. For example, the Valium® (diazepam) given to children with autism don't calm them but on the contrary, make them restless, thereby suggesting that like in epilepsy and other cerebral pathologies the intracellular chloride level is higher than the normal rate. In animal models of ASD, the NKCC1 chloride-importer inhibitor bumetanide restores physiological (Cl⁻)ᵢ levels, enhances GABAergic inhibition and attenuates electrical and behavioural symptoms of ASD. In view of the above, the inventors decided to develop bumetanide, and especially a bumetanide liquid oral formulation to improve the ASD core symptoms. The purpose of the invention is to provide a specific oral liquid formulation of bumetanide at a specific concentration suitable for the treatment of patients in need thereof and especially of a paediatric population.

The first intention was to base the study and development of said oral liquid formulation on the existing, and already approved by several drug administrations worldwide, Bumetanide Solution for Injection (Burinex®-Leo Laboratories) at the concentration of 0.5 mg/ml. Said solution for injection is composed of bumetanide, xylitol, NaH2PO₄.2H₂O (sodium phosphate monobasic monohydrate), Na₂HPO₄ (disodium phosphate) and purified water as excipients and its pH is around 6.5. One objective is to develop an oral liquid formulation containing as few excipients as possible while being well adapted to a paediatric population. Another objective is to determine the most effective posology for said paediatric population.

The main issues of the oral liquid formulation development were the solubilisation of bumetanide, the compliance to the European Pharmacopoeia (Ph. Eur.) Criteria for the efficacy of antimicrobial preservation, the masking of the bitter taste of bumetanide and the stability of the liquid oral formulation (pH and colour).

The present invention relates to a liquid oral formulation of bumetanide for use in the treatment of a patient in need thereof consisting of: an amount of 0.5 mg/ml of bumetanide, an effective amount of an antimicrobial preservative or a combination of antimicrobial preservatives, a buffering agent or a combination of buffering agents, a sweetening agent, and water.

Bumetanide is a crystalline solid that is soluble in organic solvents such as ethanol (solubility of 14 mg/ml), DMSO (solubility of 25 mg/ml), and dimethyl formamide or DMF (solubility of 33 mg/ml). Bumetanide is sparingly soluble in aqueous buffers. The solubility of bumetanide is moreover pH dependant. The storing of the aqueous solution for more than one day is not recommended. In order to solve the solubilisation of the active ingredient issue, the present application provides an oral liquid formulation of bumetanide in solution with a buffer and antimicrobial preservatives at a pH of 6.5. According to the first results, the pH level of the oral formulation was set at 6.5 as a compromise of both solubility of the active substance and efficacy of preservatives or antimicrobial agents.

The antimicrobial agents or preservatives useful in the present invention include but are not limited to sodium benzoate, sorbates, such as potassium sorbate, salts of edetate (also known as salts of ethylenediaminetetraacetic acid or EDTA, such as disodium edetate), benzaldionium chloride and parabens (such as methyl, ethyl, propyl, and butyl p-hydroxybenzoic acid esters). Antimicrobial preservatives listed above are exemplary, but each preservative must be evaluated on an experimental basis. Methods for evaluating the efficacy of preservatives in pharmaceutical formulations are known to those skilled in the art. Parabens, and especially alkyl parabens, are the presently preferred antimicrobial preservative agents. The parabens are effective over a wide pH range (4 to 8) and have a broad spectrum of antimicrobial activity. In a particular embodiment they are used in their salted form in order to speed up their dissolution in water.

In a particular embodiment, the antimicrobial preservation of the oral liquid formulation of bumetanide is ensured by a combination of parabens, more particularly methylparaben and propylparaben. In a preferred embodiment the antimicrobial preservation of the oral liquid formulation of bumetanide is ensured by a combination of salts of parabens, particularly methylparaben salt and propylparaben salt. Said antimicrobial activity is enhanced by the synergistic effect of their combination and generally increased in acidic conditions. The sodium salts of antimicrobial preservatives, and especially of parabens, allow a fast dissolution in water. The content in parabens in the oral liquid formulation of bumetanide is further established according to the Acceptable Daily Intake (ADI) for oral route and the Reflection paper on the use of methylparaben and propylparaben as excipients in human medicinal products (EMA/CHMP/SWP/272921/2012—22 Oct. 2015).

The palatability of bumetanide is a significant obstacle in developing a patient friendly oral liquid composition since bumetanide is known to have a bitter taste. This is particularly important for the paediatric populations. Said bitter taste needs to be masked and therefore the oral composition according to the invention encompasses a sweetening agent. Said sweetener or sweetening agent is selected from sucrose, fructose, dextrose, xylitol, maltitol, sorbitol, mannitol, erythritol, sucralose, aspartame, acesulfame potassium, saccharin, sodium saccharin, neotame or any derivative thereof Sucrose, fructose and dextrose are nutritive sweetener excipients with a rapid sweetness onset with short duration. Xylitol, maltitol, sorbitol, mannitol and erythrol are non-nutritive sugar alcohols with an intermediate sweetness onset with short duration and sucralose, aspartame acesulfame potassium, saccharine and neotame are non-nutritive high intensity sweeteners with variable sweetness onset with long duration. As far as a paediatric population is concerned, the inventors assessed that said sweetening agent should preferably be non-nutritive (=no caloric value) such as aspartame, sucralose, acesulfame potassium, neotame and saccharin. More preferably said sweetening agent is acesulfame potassium (or Acesulfame K or E950) since it displays a good sweetness (200 times sweeter than sucrose) with no caloric value and is stable under moderately acidic conditions and heat.

The buffering agent is selected from carbonates, citrates, gluconates, lactates, phosphates or tartrates. Said buffering agents have a low toxicity, are non-irritant and are buffered around 7.4 as pH of the human body is 7.4. The oral liquid formulation of the invention needs to be adjusted at 6.5 so the preferred buffering agents are chosen from phosphates. In a particular embodiment the buffering system is a combination of buffering agents selected from phosphates. In a preferred embodiment the buffering agents are disodium phosphate and sodium dihydrogen phosphate dihydrate.

The oral liquid formulation of bumetanide is light sensitive. Consequently, said formulation is filled in an amber glass bottle with a tamper-evident cap. A dosing pipette, consisting of a polystyrene plunger and a low-density polyethylene barrel and piston is used to administer directly the solution into the mouth of the patient in need thereof. Said dosage form is specifically developed for a paediatric population. Any other dosage form is to be considered as encompassed by the present description.

In a particular embodiment, the invention relates to a liquid oral formulation for use in the treatment of a patient in need thereof consisting of: an amount of 0.5 mg/ml of bumetanide, an effective amount of a combination of antimicrobial preservatives selected from parabens; a combination of buffering agents selected from phosphates, a sweetening agent and water. Such liquid composition can become lightly yellow when stored and this is not patient friendly especially for a paediatric population. That is why the oral liquid composition components have been further studied in order to optimize its stabilty.

In a preferred embodiment the invention relates to a liquid oral formulation for use in the treatment of a patient in need thereof consisting of: an amount of 0.5 mg/ml of bumetanide, an effective amount of sodium methylparaben and sodium propylparaben as antimicrobial preservatives; a combination of NaH₂PO₄.2H₂O and NaH₂PO₄ as buffering agents, and acesulfame potassium as sweetening agent, and water.

In another preferred embodiment the invention relates to a liquid oral formulation for use in the treatment of a patient in need thereof consisting of: an amount of 0.05 g/100 ml of bumetanide, an amount of 0,18 g/100 ml of sodium methylparaben, an amount of 0.02 g/100ml of sodium propylparaben, an amount of 0.738 g/100 ml of NaH2PO₄.2H₂O, an amount of 0.22 g/100 ml of NaH₂PO_{4,} an amount of 0.09 g/100 ml of acesulfame potassium and Quantum Satis (QS) of purified water for 100 ml.

The amount of the active ingredient needs to be adjusted according to each patient (condition, age, weight, etc.). The posology is an effective amount of the oral liquid formulation according to the invention twice a day. The dosing at 0.5 mg/ml allows a good adjustment to each patient needs. In one embodiment, the patient shall receive a dose of 1.0 mg per day, meaning one dose of 1 ml of the oral liquid formulation according to the invention twice a day. In another embodiment the patient shall receive a dose of 2.0 mg per day, meaning one dose of 2 ml of the oral liquid formulation according to the invention twice a day. In a preferred embodiment the posology is a dose of 0.5 mg to 2 mg of the oral liquid formulation twice a day for a patient in need thereof, especially a paediatric patient.

The present invention provides a method for the treatment of a population in need thereof consisting in the administration of a liquid oral formulation consisting of:
an amount of 0.5 mg/ml of bumetanide;
an effective amount of an antimicrobial preservative or a combination of antimicrobial preservatives;
a buffering agent or a combination of buffering agents;
a sweetening agent; and
water;
   twice a day.

By “population in need thereof” is meant a population with a pathology or condition selected from selected from autism and particularly autism spectrum disorder (ASD) core symptoms, tuberous sclerosis complex, fragile X syndrome, Rett syndrome, Down syndrome, cancer and particularly gliomas, spinal cord lesions, chronic pain, brain trauma, cerebrovascular infarcts and various types of epilepsies, and also acute lung injury such as pneumonias or Severe Acute Respiratory Disease due to Coronavirus-2 (SARS-CoV-2).

In a preferred embodiment the invention provides a method for the treatment of a paediatric population consisting in the administration of a liquid oral formulation consisting of: an amount of 0.5 mg/ml of bumetanide, an effective amount of an antimicrobial preservative or a combination of antimicrobial preservatives, a buffering agent or a combination of buffering agents, a sweetening agent, and water; twice a day.

Said paediatric population is a population with a pathology or condition selected from selected from autism and particularly autism spectrum disorder (ASD) core symptoms, tuberous sclerosis complex, fragile X syndrome, Rett syndrome, Down syndrome, cancer and particularly gliomas, spinal cord lesions, chronic pain, brain trauma, cerebrovascular infarcts and various types of epilepsies, and also acute lung injury such as pneumonias or Severe Acute Respiratory Disease due to Coronavirus-2 (SARS-CoV-2).

In a particular embodiment the invention provides a method for the treatment of a paediatric population with autism, and in particular with autism spectrum disorder (ASD) core symptoms, consisting in the administration of a liquid oral formulation consisting of:
an amount of 0.5 mg/ml of bumetanide;
an effective amount of a combination of antimicrobial preservatives;
a combination of buffering agents;
a sweetening agent; and
water;
   twice a day.

In a preferred embodiment the invention provides a method for the treatment of a paediatric population with autism, and in particular with autism spectrum disorder (ASD) core symptoms, consisting in the administration of a liquid oral formulation consisting of:
an amount of 0.5 mg/ml of bumetanide;
an effective amount of sodium methylparaben and sodium propylparaben as antimicrobial preservatives;
a combination of NaH₂PO₄.2H₂O and NaH₂PO₄ as buffering agents;
acesulfame potassium as sweetening agent; and
water;
   twice a day.

The posology is an oral administration of the liquid formulation according to the invention twice a day for a paediatric population. The amount of the active ingredient needs to be adjusted according to each patient (condition, age, weight, etc.).

A phase 2 clinical trial that was run with various doses of the following oral formulation of bumetanide according to the invention:
Active ingredient: bumetanide at 0.5 mg/ml 1.0 mg/ml 2.0 mg/ml and placebo
Antimicrobial preservative: sodium methylparaben and sodium propylparaben
Buffering agents: NaH₂PO₄.2H₂O and NaH₂PO₄
Sweetening agents: Acesulfame potassium and sorbitol
Solvent: water

The pH is 6.5.

The liquid oral formulation is stored in an amber glass bottle with polypropylene/low density polyethylene child-resistant and tamper-evident cap. A 1 ml dosing pipette, consisting of a polystyrene plunger and a low-density polyethylene barrel and piston was used to administer directly the solution into the mouth.

The posology was an oral administration of the liquid formulation twice a day for a paediatric population.

Detailed results of said study are available (Translational Psychiatry vol. 7, e1056 (2017)) and confirm that bumetanide can reduce the severity of ASD core symptoms in children and adolescents.

One of the observed problems of the liquid oral formulation detailed above and used during the phase 2 studies is that said formulation becomes pale yellow when stored. Said coloration is due to interactions between sorbitol and parabens.

To avoid the yellow coloration of the liquid oral formulation, several ingredients among the excipients were replaced and tested and it appears that the withdrawal of sorbitol in the combination of sweeteners eliminates said yellow coloration during storage of the bottle. The study relating to the effect of sorbitol on the stability is detailed in the example part below.

The following detailed list of ingredients is an example of a preferred oral liquid formulation according to the invention:

TABLE 1

| | Unit composition | Centesimal composition |
|---|---|---|
| Ingredient | (g) | (% m/V) |
| Bumetanide | 0.030 | 0.05 |
| Sodium methylparaben | 0.108 | 0.18 |
| Sodium propylparaben | 0.012 | 0.02 |
| NaH₂PO₄•2H₂O | 0.443 | 0.738 |
| NaH₂PO₄ | 0.132 | 0.22 |
| Acesulfame potassium | 0.054 | 0.09 |
| Purified water | QS 60 ml | QS 100 ml |

The oral liquid formulation final pH is 6.5. The solubility of bumetanide increases when pH increases but at the same time the antimicrobial activity of the parabens decreases.

The liquid oral formulation is stored in an amber glass bottle with polypropylene/low density polyethylene child-resistant and tamper-evident cap. A 1 ml dosing pipette, consisting of a polystyrene plunger and a low-density polyethylene barrel and piston is used to administer directly the solution into the mouth.

As a result the dose of 0.5 is chosen, and since the sweetening agent is mandatory for a paediatric population, the sorbitol needs to he withdrawn from the formulation for stability purposes. The stability is also ensured by the pH of 6.5.

### EXAMPLE

### Impact of Sorbitol on the Stability of the Oral Bumetanide Solution

The composition of the oral solution used at the beginning of the development is described in the table below:

TABLE 2

| Component | Centesimal formula (g/100 mL) |
|---|---|
| Bumetanide | 0.05 |
| Sodium Methyl Paraben | 0.18 |
| Sodium Propyl Paraben | 0.02 |
| NaH₂PO₄ 2H₂O | 0.738 |
| Na₂HPO₄ | 0.22 |
| Acesulfame Potassium | 0.09 |
| Sorbitol 70% | 10.00 |
| Water for injection | Qs 100 mL |

During stability studies, the following modifications were observed: coloring of the solution and apparition of degradation compounds

A study has been performed to explain these observations.

To assess the situation, three solutions were prepared:
solution A corresponding to the solution described in the table above,
solution B corresponding to solution A without acesulfame and without sorbitol, and
solution C corresponding to solution A without sorbitol.

The detailed composition of he three solutions is described in he table 3 below.

TABLE 3

| Composition of the | Solution | Solution | Solution |
|---|---|---|---|
| 3 solutions (g/100 mL) | A | B | C |
| Bumetanide | 0.05 | 0.05 | 0.05 |
| Sodium Methyl Paraben | 0.18 | 0.18 | 0.18 |
| Sodium Propyl Paraben | 0.02 | 0.02 | 0.02 |
| NaH₂PO₄ 2H₂O | 0.738 | 0.738 | 0.738 |
| Na₂HPO₄ | 0.22 | 0.22 | 0.22 |
| Acesulfame Potassium | 0.09 | — | 0.09 |
| Sorbitol 70% | 10.00 | — | — |
| Water for injection | Qs 100 mL | Qs 100 mL | Qs 100 mL |

The solutions have been stored in closed bottles at 55° C.-60° C. for 1 month.

After 1 month the coloring and the degradation compounds have been checked.

The color of the solution has been checked according to the European Pharmacopoeia method (Ph.Eur 2.2.2), The degradation compounds have been quantified by HPLC.

### Results

The results are described in the table 4 below:

TABLE 4

| | Solution A | | Solution B | | Solution C | |
|---|---|---|---|---|---|---|
| | | 1 month | | 1 month | | 1 month |
| | T0 | at 55-60 | T0 | at 55-60 | T0 | at 55-60 |
| Coloration | <B9 | <B3 | <B9 | <B9 | <B9 | <B9 |
| | | Yellow | | | | |
| | | brown | | | | |
| | | solution | | | | |
| Total Degradation | — | 1.01% | — | ND ⁽*⁾ | — | ND ⁽*⁾ |
| compounds | | | | | | |

⁽*⁾ ND = No degradation compound detected (limit of detection = 0.02%)

The presence of sorbitol is clearly responsible for the stability issues observed with formulation used at the beginning of the development.

As a conclusion the withdrawal of the sorbitol is mandatory for the stability of the oral liquid formulation according to the invention.

1. A liquid oral formulation for use in the treatment of a patient in need thereof consisting of:
an amount of 0.5 mg/ml of bumetanide;
an effective amount of an antimicrobial preservative or a combination of antimicrobial preservatives;
a buffering agent or a combination of buffering agents;
a sweetening agent; and
water.

2. The formulation of claim 1 wherein the antimicrobial preservative is selected from sodium benzoate, sorbate, salt of edetate, benzaldionium chloride and paraben, salts thereof or a combination thereof.

3. The formulation of claim 1, wherein the antimicrobial preservative is selected from alkyl paraben salts, and preferably a combination of alkyl paraben salts.

4. The formulation of claim 3 wherein the combination of antimicrobial preservative compounds consists in sodium methyl paraben combined with sodium propyl paraben.

5. The formulation of claim 1, wherein the buffering agent is selected from carbonates, citrates, gluconates, lactates, phosphates or tartrates, or a combination thereof.

6. The formulation of claim 5, wherein the buffering agent is a combination of sodium dihydrogen phosphate dihydrate and disodium phosphate.

7. The formulation of claim 1, wherein the sweetening agent is selected from sucrose, fructose, dextrose, xylitol, maltitol, sorbitol, mannitol, erythritol, sucralose, aspartame, acesulfame potassium, saccharin, sodium saccharin, neotame or any derivative thereof.

8. The formulation of claim 7, wherein the sweetening agent is selected from sucralose, aspartame, acesulfame potassium, saccharin, sodium saccharin and neotame.

9. The formulation according to claim 7, wherein the sweetening agent is acesulfame potassium.

10. The formulation according to claim 1, wherein its pH is 6.5.

11. The formulation according to claim 1 for use twice a day in the treatment of a pathology or condition selected from autism, Fragile X syndrome, tuberous sclerosis complex, Rett syndrome, Down syndrome, cancer and particularly gliomas, spinal cord lesions, chronic pain, brain trauma, cerebrovascular infarcts and various types of epilepsies and also acute lung injury such as pneumonia or Severe Acute Respiratory Disease due to Coronavirus-2 (SARS-CoV-2), and more preferably in the treatment of autism and particularly for the improvement of autism spectrum disorder (ASD) core symptoms.

12. The formulation of claim 11 wherein the patient is a child.

13. A method for the treatment of a paediatric population in need thereof comprising the administration of a liquid oral formulation consisting of:
an amount of 0.5 mg/ml of bumetanide;
an effective amount of an antimicrobial preservative or a combination of antimicrobial preservatives;
a buffering agent or a combination of buffering agents;
a sweetening agent; and
water;
twice a day.

14. The method of treatment of claim 13 wherein the paediatric population in need thereof is a population with a pathology or condition selected from autism and particularly autism spectrum disorder (ASD) core symptoms, Fragile X syndrome, tuberous sclerosis complex, Rett syndrome, Down syndrome, cancer and particularly gliomas, spinal cord lesions, chronic pain, brain trauma, cerebrovascular infarcts and various types of epilepsies and also acute lung injury such as pneumonia or Severe Acute Respiratory Disease due to Coronavirus-2 (SARS-CoV-2).

15. The method of treatment of claim 13 wherein the pathology or condition is autism.
